(19) United States
(12) Patent Application Publication
(10) Pub. No.: US 2024/0177420 A1
(43) Pub. Date: May 30, 2024

(54) HUMAN MODEL RECOVERY BASED ON VIDEO SEQUENCES

(71) Applicant: Shanghai United Imaging Intelligence Co., Ltd., Shanghai (CN)

(72) Inventors: Srikrishna Karanam, Bangalore (IN); Runze Li, South San Francisco, CA (US); Meng Zheng, Cambridge, MA (US); Ziyan Wu, Lexington, MA (US)

(73) Assignee: Shanghai United Imaging Intelligence Co., Ltd., Shanghai (CN)

(21) Appl. No.: 17/994,675

(22) Filed: Nov. 28, 2022

(51) Int. Cl. G06T 17/20; A61B 34/10; G06T 7/73

(52) U.S. Cl. CPC G06T 17/20; A61B 34/10; G06T 7/75; A61B 2034/105; G06T 2207/30196

(57) ABSTRACT

A video sequence depicting a person in a medical environment may be obtained and used for determining one or more human models of the person. A first human model representing a first pose or a first body shape of the person may be determined based on a first subset of images from the video sequence, while a second human model representing a second pose or a second body shape of the person may be determined based on a second subset of images from the video sequence. The second 2D or 3D representation of the person may include an adjustment to the first 2D or 3D representation of the person based on the observation of the person provided by the second subset of images.

### BACKGROUND

A two-dimensional (2D) or three-dimensional (3D) representation of a patient's body (e.g., a human model such as a human mesh) that realistically reflects the individual patient's body shape and/or pose may be used in a variety of medical applications including patient positioning, surgical navigation, unified medical record analysis, etc. For example, with radiation therapy and medical imaging, success often hinges upon the ability to place and maintain a patient in a desirable position so that the treatment or scan can be performed in a precise and accurate manner. Having real time knowledge about an individual patient's physical characteristics such as the patient's body shape and/or pose in these situations may bring many benefits including, for example, faster and more accurate positioning of the patient in accordance with a scan or treatment protocol, more consistent results, etc. In other example situations, the real time knowledge about an individual patient's physical characteristics and/or movement in a medical environment may also provide means for generating the control signals needed to automatically adjust the operating parameters of medical equipment (e.g., height of a patient bed) so as to accommodate the patient's physical characteristics.

### SUMMARY

Conventional methods and systems for generating representations of a patient's body may not accurately represent the patient's body shape and/or pose with respect to all of the patient's body parts. Described herein are systems, methods and instrumentalities for generating and/or adjusting a 2D or 3D representation of a person based on a video sequence of the person. The systems, methods and/or instrumentalities may utilize one or more processors configured to obtain a video sequence depicting positions, poses, and/or movements of the person in a medical environment. The processor(s) may be further configured to determine a first two-dimensional (2D) or three-dimensional (3D) representation of the person based on at least a first subset of images from the video sequence, wherein the first 2D or 3D representation of the person may represent a first pose or a first body shape of the person in the medical environment. The processor(s) may be additionally configured to determine a second 2D or 3D representation of the person based on at least a second subset of images from the video sequence, wherein the second 2D or 3D representation of the person may represent a second pose or a second body shape of the person in the medical environment. The second 2D or 3D representation of the person may include an adjustment (e.g., an improvement) to the first 2D or 3D representation of the person, for example, based on an observation of the person provided by the second subset of images that may not be provided by the first subset of images.

In example implementations of the present disclosure, the adjustment included in the second 2D or 3D representation of the person may include a depiction of a body part of the person that is missing from the first 2D or 3D representation of the person. Furthermore, the second 2D or 3D representation of the person may be determined further based on the first 2D or 3D representation of the person. In example implementations of the present disclosure, the one or more processors described herein may be further configured to determine a body part of the person that is missing from the first 2D or 3D representation of the person, and reconstruct the body part in the second 2D or 3D representation of the person based on the second subset of images. The one or more processors may be further configured to determine the at least one of the first 2D or 3D representation of the person or the second 2D or 3D representation of the person based on a machine-learning (ML) model, which may be learned and/or implemented using a convolutional neural network or a recurrent neural network.

In example implementations of the present disclosure, the one or more processors described herein may be further configured to determine at least one of the first 2D or 3D representation of the person or the second 2D or 3D representation of the person based on multiple machine-learning (ML) models, wherein a first one of the multiple ML models may be trained for predicting a representation of a first body part of the person based on the video sequence, a second one of the multiple ML models may be trained for predicting a representation of a second body part of the person based on the video sequence, and the at least one of the first 2D or 3D representation of the person or the second 2D or 3D representation of the person may be determined by combining the representation of the first body part of the person and the representation of the second body part of the person.

In example implementations of the present disclosure, at least one of the first 2D or 3D representation of the person or the second 2D or 3D representation of the person includes a 3D mesh model of the person. Furthermore, the 3D mesh model of the person may include a first plurality of parameters associated with a pose of the person, and a second plurality of parameters associated with a body shape of the person. In example implementations of the present disclosure, the one or more processors described herein may obtain the video sequence from an image capturing device that may include at least one of a red-green-blue (RGB) image sensor, a depth sensor, an infrared sensor, a radar sensor, or a pressure sensor. The one or more processors described herein may be further configured to provide the second 2D or 3D representation of the person to a receiving device for adjusting a medical device in the medical environment.

### BRIEF DESCRIPTION OF THE DRAWINGS

A more detailed understanding of the examples disclosed herein may be had from the following description, given by way of example in conjunction with the accompanying drawings.

FIG. 1 is a diagram illustrating an example environment in which the methods and instrumentalities disclosed herein may be used to generate and/or adjust an estimated 2D or 3D human model.

FIG. 2 is a simplified block diagram illustrating an example of using a machine learning (ML) model to recover a 2D or 3D human model for a person based on a video sequence depicting the person in a medical environment.

FIG. 3 is a simplified block diagram illustrating an example of recovering a 2D or 3D human model based on a video sequence.

FIG. 4 is a simplified block diagram illustrating an example of recovering a 2D or 3D human model based on a video sequence.

FIG. 5 is a flow diagram illustrating example operations that may be associated with recovering a 2D or 3D human model based on a video sequence.

FIG. 6 is a flow diagram illustrating example operations that may be associated with training a neural network for performing one or more of the tasks described herein.

FIG. 7 is a simplified block diagram illustrating example components of an apparatus that may be used to perform one or more of the tasks described herein.

### DETAILED DESCRIPTION

The present disclosure is illustrated by way of example, and not by way of limitation, in the figures of the accompanying drawings.

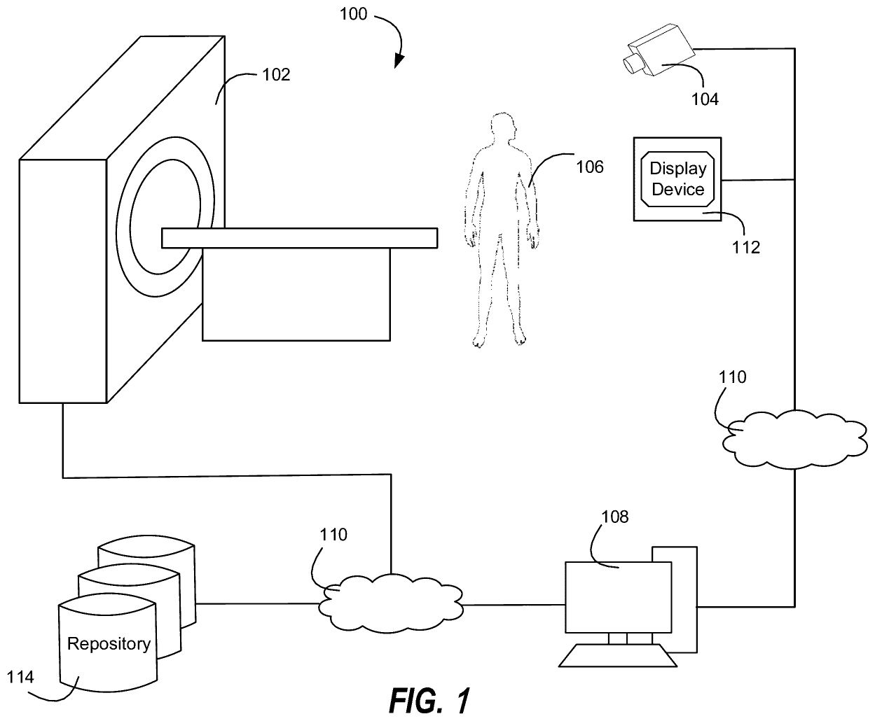

FIG. 1 is a diagram illustrating an example of a medical environment 100 that may utilize the methods and instrumentalities disclosed herein to generate and/or adjust an estimated 2D or 3D body model. As shown in the figure, the environment 100 may be configured to provide a medical scan or imaging procedure based on a medical scanner 102 (e.g., a computed tomography (CT) scanner, a magnetic resonance imaging (MRI) machine, a positron emission tomography (PET) scanner, an X-ray machine, etc.), even though the environment 100 may also be adapted to provide other types of healthcare services including, for example, radiation therapy, surgery, etc.

The environment 100 may include at least one sensing device 104 (e.g., an image capturing device) configured to capture a video sequence (e.g., comprising multiple images) of a patient 106 (or another person such as a medical professional) while the patient stays in or moves about the environment 100 (e.g., standing in front of the medical scanner 102, lying down on a scan or treatment bed, moving from a location to another location, etc.). The sensing device 104 may comprise one or more sensors including one or more cameras (e.g., digital cameras), one or more red, green and blue (RGB) sensors, one or more depth sensors, one or more RGB plus depth (RGB-D) sensors, one or more thermal sensors such as infrared (FIR) or near-infrared (NIR) sensors, one or more radar sensors, and/or the like. In example implementations, the sensing device 104 may be installed or placed at a location of the environment 100 (e.g., a ceiling, a door frame, etc.) from which a view of the patient may be obtained so as to capture the position, pose, body shape, and/or movement of the patient inside the environment 100.

The sensing device 104 may include one or more processors configured to process the video sequence of the patient 106 captured by the sensors described herein. Additionally, or alternatively, the sensing device 104 may transmit the video sequence to a processing unit 108 (e.g., a computer) associated with the environment 100 for processing. The processing unit 108 may be communicatively coupled to the sensing device 104, for example, via a communication network 110, which may be a wired or wireless communication network. In response to receiving the images of the patient 106, the sensing device 104 and/or the processing device 108 may analyze the images (e.g., at a pixel level) to determine various anatomical characteristics of the patient 106 (e.g., shape, pose, etc.).

In response to obtaining the video sequence of the patient 106, the sensing device 104 and/or the processing unit 108 may analyze the video sequence to identify visual features that may be associated with various body keypoints (e.g., joint locations) of the patient 106, and generate and/or refine a 2D or 3D human model for the patient based on the identified body keypoints. For example, the sensing device 104 and/or the processing unit 108 may be configured to determine a first 2D or 3D human model for the patient 106 based on a first subset of images from the video sequence, where the first 2D or 3D human model may represent a first pose or a first body shape of the patient in the environment 100. In addition, the sensing device 104 and/or the processing unit 108 may be further configured to determine a second 2D or 3D human model of the patient based on a second subset of images from the video sequence, where the second 2D or 3D human model may represent a second pose or a second body shape of the person in the environment 100, and where the second 2D or 3D human model may include an adjustment to the first 2D or 3D human model. Thus, if a body keypoint of the patient 106 is occluded or blocked in the first subset of images, but is visible in the second subset of images, the second 2D or 3D human model may be used to compensate (e.g., correct or improve) the first 2D or 3D human model. Additionally, a constraint may be imposed to ensure consistency among the 2D or 3D human models generated based on the different subsets of images such that a more accurate human model may be recovered based on the video sequence (e.g., which may include multiple observations of the patient). Further, by recording the movements of the patient 106 in the video sequence, dynamic information about the patient 106 such as how the patient moves about the environment 100 may be determined based on the recovered human model(s).

The sensing device 104 and/or the processing unit 108 may be configured to perform one or more of the operations described herein, for example, based on a machine-learning (ML) model learned and/or implemented using an artificial neural network. The 2D or 3D body model of the patient 106 may include a non-parametric model, or a parametric model such as a skinned multi-person linear (SMPL) model, that may indicate the shape, pose, and/or other anatomical characteristics of the patient 106. Once generated, the 2D or 3D human model may be used to facilitate a plurality of medical applications and services including, for example, patient positioning, patient monitoring, medical protocol design, unified or correlated diagnoses and treatments, surgical navigation, etc. For example, the processing device 108 may determine, based on the 3D human model, whether the position and/or pose of the patient 106 meets the requirements of a predetermined protocol (e.g., while the patient 106 is standing in front of the medical scanner 102 or lying down on a scan bed), and provide confirmation or adjustment instructions (e.g., via the display device 112), to help the patient 106 get into the desired position and/or pose. The processing unit 108 may also control (e.g., adjust) one or more operating parameters of the medical scanner 102 such as the height of the scan bed based on the body shape and/or pose of the patient 106 as indicated by the 2D or 3D body model. In this way, the environment 100 may be automated (e.g., at least partially) to protect itself against obvious errors with no or minimum human intervention.

As another example, the sensing device 104 and/or the processing unit 108 may be coupled with a medical record repository 114 (e.g., one or more stand-alone or cloud based data storage devices) configured to store patient medical records including scan images of the patient 106 obtained through other imaging modalities (e.g., CT, MR, X-ray, SPECT, PET, etc.). The processing unit 108 may be configured to analyze the medical records of patient 106 stored in the repository in storage device(s) 114 using the 2D or 3D human model as a reference so as to obtain a comprehensive understanding of the medical conditions of patient 106. For instance, the processing unit 108 may be configured to align scan images of the patient 106 from the repository 114 with the 2D or 3D human model to allow the scan images to be presented (e.g., via display device 112) and/or analyzed with respect to the anatomical characteristics (e.g., body shape and/or pose) of the patient 106 as indicated by the 2D or 3D human model.

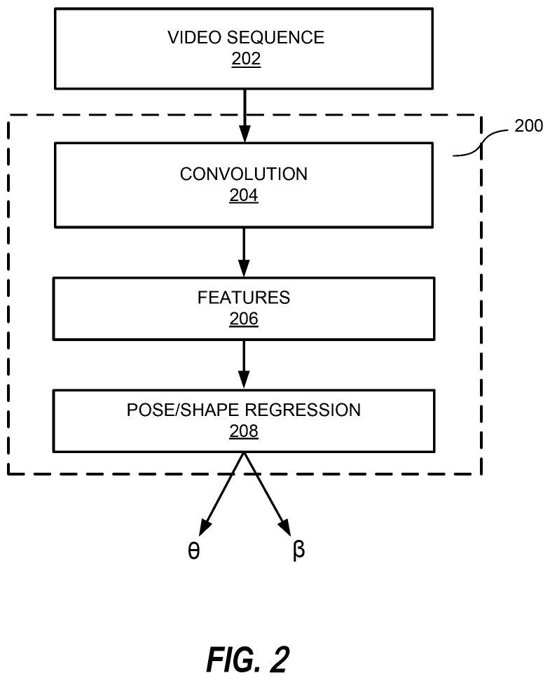

FIG. 2 shows a simplified block diagram illustrating how a machine learning (ML) model 200 may be used to recover a 2D or 3D human model based on a video sequence that may include multiple observations or views of a person (e.g., a patient, a doctor, etc.) in a medical environment. As shown, given a video sequence 202 of the person (e.g., patient 106 of FIG. 1), a plurality of features 206 may be extracted from the video sequence 202, for example, by performing a series of convolution operations 204 using an artificial neural network. The extracted features (e.g., represented in a feature vector or feature map) may be used to predict a set of body keypoints (e.g., joint locations) of the person that may then be provided to a pose/shape regression module 208 to infer parameters for recovering/estimating the 2D or 3D human model. The inferred parameters may include, for example, one or more pose parameters θ 210 and/or one or more shape parameters β 212 that may respectively indicate the pose and shape of the individual person as shown in the video sequence 202.

The artificial neural network may include a convolutional neural network (CNN) and/or a recurrent neural network (RNN) that in turn may include multiple layers such as an input layer, one or more convolutional layers (e.g., associated with linear or non-linear activation functions), one or more pooling layers, one or more fully connected layers, and/or an output layer. Each of the aforementioned layers may include a plurality of filters (e.g., kernels) and each filter may be designed to detect (e.g., learn) features associated with a body keypoint of the person. The filters may be associated with respective weights that, when applied to an input, produce an output indicating whether certain visual features (e.g., features 206) have been detected. The weights associated with the filters may be learned by the neural network through a training process that may include inputting a large number of images from a training dataset to the neural network, predicting a result (e.g., features and/or body keypoint) using presently assigned parameters of the neural network, calculating a difference or loss (e.g., based on mean squared errors (MSE), L1/L2 norm, etc.) between the prediction and a corresponding ground truth, and updating the parameters (e.g., weights assigned to the filters) of the neural network so as to minimize the difference or loss (e.g., based on a stochastic gradient descent of the loss). Once trained (e.g., having learned to recognize the features associated with the body keypoints in the training images), the neural network may receive a video sequence of the person at an input, process each image in the video sequence to determine the respective locations of a set of body keypoints of the person in the image, and regress the pose parameters θ 210 and/or shape parameters β 212 based on the locations of the body keypoints, before using the parameters to recover a 3D human model (e.g., an SMPL model) of the person. For example, the neural network (e.g., ML model) may be used to identify 23 joint locations of a skeletal rig of the patient as well as a root joint of the patient, from which 72 pose-related parameters θ (e.g., 3 parameters for each of the 23 joints and 3 parameters for the root joint) may be inferred. In addition, a principal component analysis (PCA) may be performed for one or more images (e.g., for each image) in the video sequence to derive a set of PCA coefficients, the first 10 coefficients of which may be used as the shape parameters β. Based on these parameters, a plurality of vertices (e.g., 6890 vertices based on 72 pose parameters and 10 shape parameters) may be determined and used to reconstruct a 3D mesh of the person's body, for example, by connecting multiple vertices with edges to form a polygon (e.g., such as a triangle), connecting multiple polygons to form a surface, using multiple surfaces to determine a 3D shape, and applying texture and/or shading to the surfaces and/or shapes.

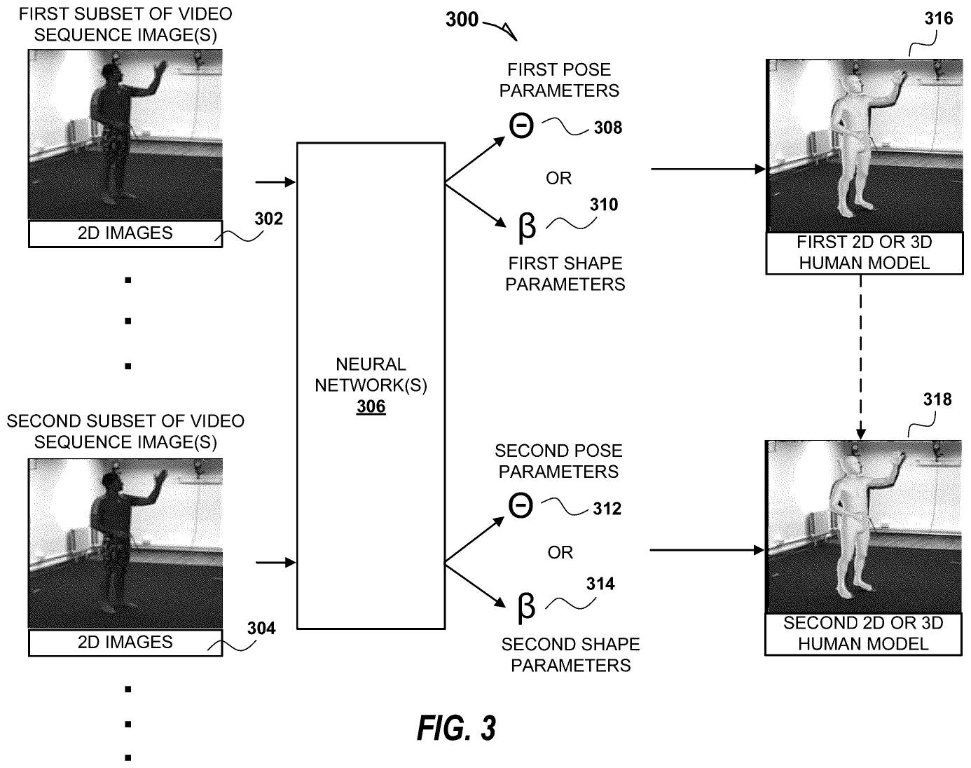

FIG. 3 shows a simplified block diagram illustrating a data flow 300 for recovering one or more 2D or 3D representations (e.g., 2D or 3D human models) of a person based on a video sequence depicting the position, pose, and/or movements of the person in a medical environment. An apparatus, comprising one or more processors, may be configured to obtain the video sequence and determine a first 2D or 3D representation 316 (e.g., a first human model) of the person based on at least a first subset 302 of images from the video sequence, wherein the first 2D or 3D representation 316 of the person may represent a first pose and/or a first body shape of the person in the medical environment. The apparatus may be further configured to determine a second 2D or 3D representation 318 (e.g., a second human model) of the person based on at least a second subset 304 of images from the video sequence, wherein the second 2D or 3D representation 318 of the person may represent a second pose or a second body shape of the person in the medical environment. The second 2D or 3D representation 318 of the person may include an adjustment to the first 2D or 3D representation 316 of the person, for example, if one or more body keypoints of the person are blocked, occluded, or otherwise invisible in the first subset of images, but are visible in the second subset of images (e.g., due to movements of the person from a first position in the medical environment to a second position in the medical environment). In other examples, even if there is no occlusion or blockage in the first subset of images or the second subset of images, the second 2D or 3D representation 318 of the person may still be used to improve the accuracy of the first 2D or 3D representation 316 of the person, for example, by aggregating (e.g., averaging) the pose and/or shape parameters predicted using the first and second subsets of images, and predicting the second 2D or 3D representation 318 of the person based on the aggregated parameters. In yet another set of examples, a consistency constraint may be imposed on the first and second 2D or 3D representations of the person to ensure that the two representations complement but do not contradict each other. For instance, while the second 2D or 3D representation 318 of the person may fill in gaps left in the first 2D or 3D representation 316 of the person (e.g., due to occlusions or blockage), existing body keypoints in the first 2D or 3D representation 316 may not be contradicted by body keypoints in the second 2D or 3D representation 318. In examples, if there is conflict information provided by the first representation 316 and the second representation 318, respective confidence scores may be determined (e.g., by the ML model or neural network described herein) for the first and second representations, and priority may be given to the representation with a higher confidence score.

The first subset 302 of images from the video sequence and the second subset 304 of images from the video sequence may comprise one or more 2D image(s). These 2D images may be captured, for example, by the sensing device 104 of FIG. 1. As shown, given the 2D input image(s) of the person (e.g., patient 106 of FIG. 1) from subsets 302 and 304 of images from the video sequence, first pose parameters θ 308 and/or first shape parameters β 310, and second pose parameters θ 312 and/or second shape parameters β 314, may be extracted from the 2D image(s), for example, using one or more neural network(s) 306 (e.g., the CNN or RNN described herein) to process the 2D images.

As shown, the first 2D or 3D human model 316 may be generated based on the first pose parameters θ 308 and/or the first shape parameters β 310 that may respectively indicate the pose and/or shape of the individual person's body during the portion of the video sequence from which the first subset 302 of images from the video sequence were taken. Furthermore, the second 2D or 3D human model 318 may be generated based on the second pose parameters θ 312 and/or the second shape parameters β 314 that may respectively indicate the pose and/or shape of the individual person's body during the portion of the video sequence from which the second subset 304 of images from the video sequence were taken. As noted above, the second 2D or 3D human model 318 may include an adjustment of the first 2D or 3D human model 316 (e.g., changed, added, or removed characteristics to the model representation of the person). In some embodiments of the present disclosure, the adjustment included in the second 2D or 3D representation 318 of the person may include a depiction of a body part (e.g., a body keypoint such as a joint location) of the person that may be missing from the first 2D or 3D representation 316 of the person. Furthermore, the second 2D or 3D representation 318 of the person may be provided to a receiving device for controlling (e.g., adjusting) one or more operating parameters of medical equipment (e.g., the height of the scan bed of the medical scanner 102) based on the body shape and/or pose of the person indicated by the second 2D or 3D representation 318 of the person.

In some example embodiments, the second 2D or 3D representation 318 of the person may be determined further based on the first 2D or 3D representation 316 of the person. For example, the one or more processors of processing device 108 of FIG. 1 may be configured to duplicate existing body parts represented by the first 2D or 3D representation 316 of the person in the second 2D or 3D representation 318, and further add (e.g., reconstruct) a body part of the person that is missing from the first 2D or 3D representation 316 of the person to the second 2D or 3D representation 318 of the person based on the second subset 304 of images that may depict the missing body part.

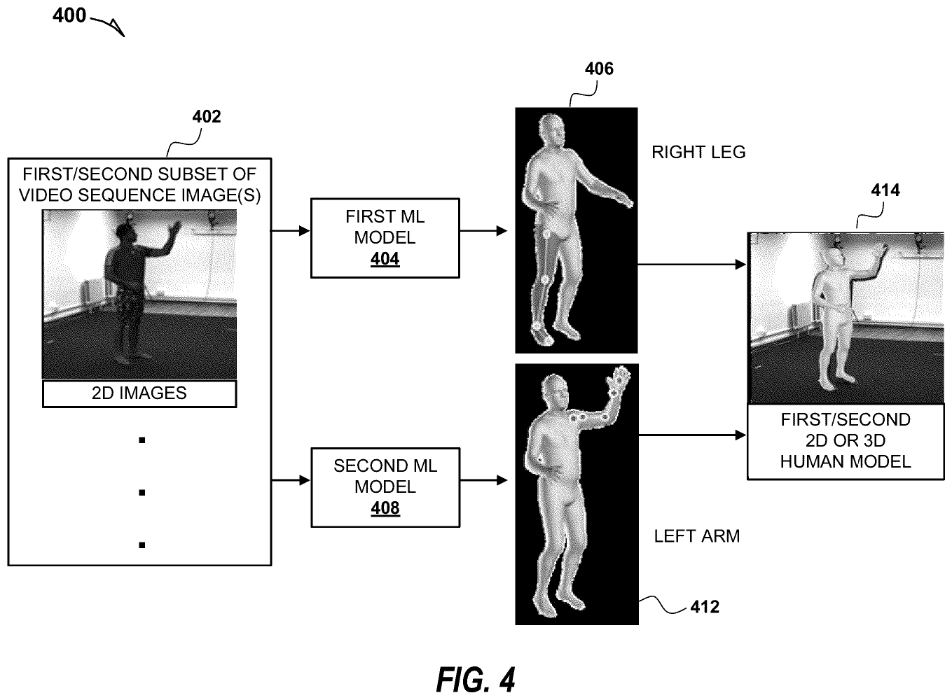

FIG. 4 shows a simplified block diagram illustrating an example of recovering a 2D or 3D human model based on a subset of images from a video sequence by using machine-learning (ML) models. A first machine learning model 404 of the multiple machine learning models may be trained for predicting a representation 406 of a first body part of the person (e.g., the right leg) based on at least one subset 402 of the first and/or second subsets of images from the video sequence (e.g., subsets 302 and/or 304 of FIG. 3), and a second machine learning model 408 of the multiple machine learning models may be trained for predicting a representation 412 of a second body part of the person (e.g., the left arm) based on the at least one subset 402 of the first and/or second subsets of images from the video sequence (e.g., subsets 302 and/or 304 of FIG. 3). A human model 414 of the person may then be determined by combining at least the representation 406 of the first body part of the person (e.g., the right leg) and the representation 412 of the second body part of the person (e.g., the left arm). Relationships between the determined first body part, second body part, and other body parts of the person (e.g., as indicated by a human kinetic chain) may be leveraged to improve the accuracy of the human model 414.

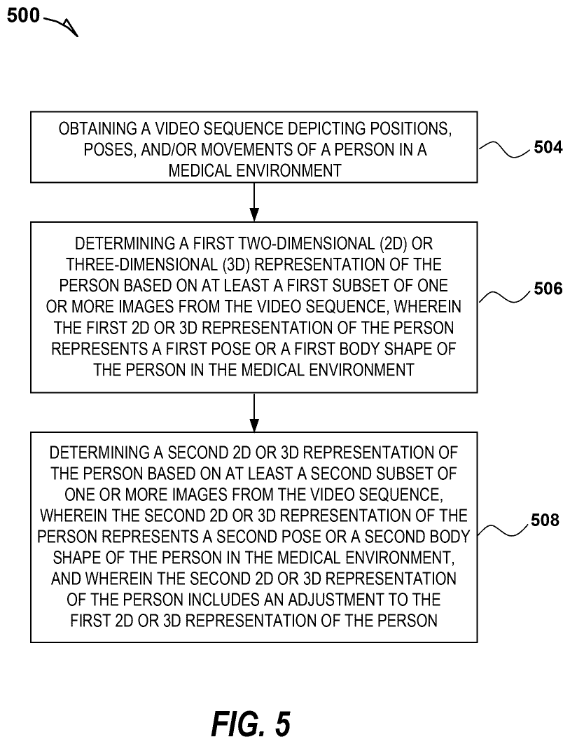

FIG. 5 shows a flow diagram illustrating an example method 500 for recovering a 2D or 3D human model based on a video sequence depicting the positions, poses, and/or movements of a person in a medical environment.

The method 500 may including obtaining, at 504, a video sequence depicting positions, poses, and/or movements of the person (e.g., patient 106 of FIG. 1) in the medical environment (e.g., environment 100 of FIG. 1). At 506, a first 2D or 3D representation (e.g., model 316 of FIG. 3) of the person may be determined based on at least a first subset of images from the video sequence (e.g., subset 302 of FIG. 3), wherein the first 2D or 3D representation of the person may represent a first pose and/or a first body shape of the person in the medical environment (e.g., as indicated by first pose parameters θ 308 and/or first shape parameters β 310 of FIG. 3). At 508, a second 2D or 3D representation (e.g., model 318 of FIG. 3) of the person may be determined based on at least a second subset of images from the video sequence (e.g., subset 304 of FIG. 3), wherein the second 2D or 3D representation of the person may represent a second pose or a second body shape of the person in the medical environment (e.g., as described by second pose parameters θ 312 and/or second shape parameters β 314 of FIG. 3), and wherein the second 2D or 3D representation (e.g., 318) of the person may include an adjustment (e.g., improvement) to the first 2D or 3D representation (e.g., 316) of the person.

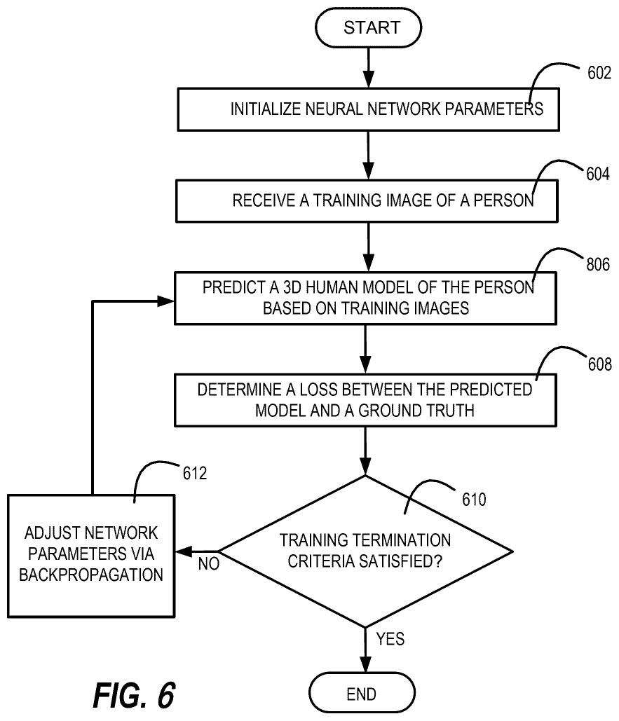

FIG. 6 illustrates example operations that may be associated with training a neural network (e.g., an ML model implemented by the neural network) for performing one or more of the tasks described herein. As shown, the training operations may include initializing the operating parameters of the neural network (e.g., weights associated with various layers of the neural network) at 602, for example, by sampling from a probability distribution or by copying the parameters of another neural network having a similar structure. The training operations may further include processing an input (e.g., a training image) using presently assigned parameters of the neural network at 604, and making a prediction for a desired result (e.g., a feature vector, pose and/or shape parameters, a human model, etc.) at 606. The prediction result may then be compared to a ground truth at 608 to determine a loss associated with the prediction based on a loss function such as mean squared errors between the prediction result and the ground truth, an L1 norm, an L2 norm, etc. The loss thus calculated may be used to determine, at 610, whether one or more training termination criteria are satisfied. For example, the training termination criteria may be determined to be satisfied if the loss is below a threshold value or if the change in the loss between two training iterations falls below a threshold value. If the determination at 610 is that the termination criteria are satisfied, the training may end; otherwise, the presently assigned network parameters may be adjusted at 612, for example, by backpropagating a gradient descent of the loss function through the network before the training returns to 606.

For simplicity of explanation, the operations of the methods are depicted and described herein with a specific order. It should be appreciated, however, that these operations may occur in various orders, concurrently, and/or with other operations not presented or described herein. Furthermore, it should be noted that not all operations that the apparatus is capable of performing are depicted in the drawings or described herein. It should also be noted that not all illustrated operations may be required to be performed.

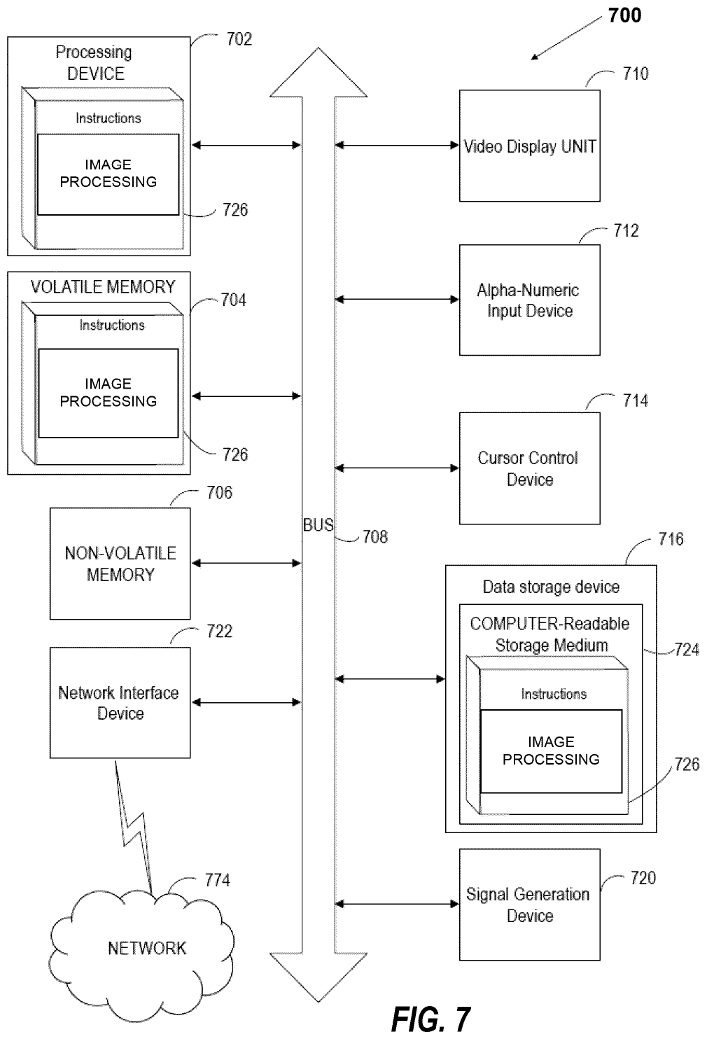

The systems, methods, and/or instrumentalities described herein may be implemented using one or more processors, one or more storage devices, and/or other suitable accessory devices such as display devices, communication devices, input/output devices, etc. FIG. 7 shows a simplified block diagram illustrating an example apparatus 700 that may be configured to perform the tasks described herein. In embodiments, apparatus 700 may be connected (e.g., via a network, such as a Local Area Network (LAN), an intranet, an extranet, or the Internet) to other computer systems. Apparatus 700 may operate in the capacity of a server or a client computer in a client-server environment, or as a peer computer in a peer-to-peer or distributed network environment. Apparatus 700 may include a personal computer (PC), a tablet PC, a set-top box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” may include any collection of computers that individually or jointly execute a set (or multiple sets) of instructions to perform any one or more of the tasks described herein.

Furthermore, apparatus 700 may include a processing device 702 (e.g., the sensing device 104 of FIG. 1 and/or the processing unit 108 of FIG. 1), a volatile memory 704 (e.g., random access memory (RAM)), a non-volatile memory 706 (e.g., read-only memory (ROM) or electrically-erasable programmable ROM (EEPROM)), and a data storage device 716, which may communicate with each other via a bus 708. Processing device 702 may be provided by one or more processors such as a general purpose processor (such as, for example, a complex instruction set computing (CISC) microprocessor, a reduced instruction set computing (RISC) microprocessor, a very long instruction word (VLIW) microprocessor, a microprocessor implementing other types of instruction sets, or a microprocessor implementing a combination of types of instruction sets) or a specialized processor (such as, for example, an application specific integrated circuit (ASIC), a field programmable gate array (FPGA), a digital signal processor (DSP), or a network processor).

Apparatus 700 may further include a network interface device 722, a video display unit 710 (e.g., an LCD), an alphanumeric input device 712 (e.g., a keyboard), a cursor control device 714 (e.g., a mouse), and/or a signal generation device 720. Data storage device 716 may include a non-transitory computer-readable storage medium 724 on which to store instructions 726 encoding any one or more of the image processing methods or functions described herein. Instructions 726 may also reside, completely or partially, within volatile memory 704 and/or within processing device 702 during execution thereof by system 700, hence, volatile memory 704 and processing device 702 may comprise machine-readable storage media.

While computer-readable storage medium 724 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein.

The methods, components, and characteristics described herein may be implemented by discrete hardware components or may be integrated in the functionality of other hardware components such as ASICS, FPGAs, DSPs or similar devices. In addition, the methods, components, and characteristics may be implemented by firmware modules or functional circuitry within hardware devices. Further, the methods, components, and characteristics may be implemented in any combination of hardware devices and computer program components, or in computer programs.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing.” “determining.” “enabling.” “identifying.” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
one or more processors configured to:
obtain a video sequence depicting movements of a person in a medical environment;
determine a first two-dimensional (2D) or three-dimensional (3D) representation of the person based on at least a first subset of images from the video sequence, wherein the first 2D or 3D representation of the person represents a first pose or a first body shape of the person in the medical environment; and
determine a second 2D or 3D representation of the person based on at least a second subset of images from the video sequence, wherein the second 2D or 3D representation of the person represents a second pose or a second body shape of the person in the medical environment, and wherein the second 2D or 3D representation of the person includes an adjustment to the first 2D or 3D representation of the person.

2. The apparatus of claim 1, wherein the adjustment included in the second 2D or 3D representation of the person includes a depiction of a body part of the person that is missing from the first 2D or 3D representation of the person.

3. The apparatus of claim 1, wherein the second 2D or 3D representation of the person is determined further based on the first 2D or 3D representation of the person.

4. The apparatus of claim 3, wherein the one or more processors are configured to determine a body part of the person that is missing from the first 2D or 3D representation of the person, and reconstruct the body part in the second 2D or 3D representation of the person based on the second subset of images.

5. The apparatus of claim 1, wherein at least one of the first 2D or 3D representation of the person or the second 2D or 3D representation of the person is determined based on a machine-learning (ML) model.

6. The apparatus of claim 5, wherein the one or more processors are configured to implement the ML model via a convolutional neural network or a recurrent neural network.

7. The apparatus of claim 1, wherein at least one of the first 2D or 3D representation of the person or the second 2D or 3D representation of the person is determined based on multiple machine-learning (ML) models, a first one of the multiple ML models trained for predicting a representation of a first body part of the person based on the video sequence, a second one of the multiple ML models trained for predicting a representation of a second body part of the person based on the video sequence, and wherein the at least one of the first 2D or 3D representation of the person or the second 2D or 3D representation of the person is determined by combining at least the representation of the first body part of the person and the representation of the second body part of the person.

8. The apparatus of claim 1, wherein at least one of the first 2D or 3D representation of the person or the second 2D or 3D representation of the person includes a 3D mesh model of the person.

9. The apparatus of claim 8, wherein the 3D mesh model of the person includes a first plurality of parameters associated with a pose of the person, the 3D mesh model further including a second plurality of parameters associated with a body shape of the person.

10. The apparatus of claim 1, wherein the video sequence is captured by a single image capturing device that includes at least one of a red-green-blue (RGB) image sensor, a depth sensor, an infrared sensor, a radar sensor, or a pressure sensor.

11. The apparatus of claim 1, wherein the one or more processors are further configured to provide the second 2D or 3D representation of the person to a receiving device for adjusting a medical device in the medical environment.

12. A method, comprising:
obtaining a video sequence depicting movements of a person in a medical environment;
determining a first two-dimensional (2D) or three-dimensional (3D) representation of the person based on at least a first subset of images from the video sequence, wherein the first 2D or 3D representation of the person represents a first pose or a first body shape of the person in the medical environment; and
determining a second 2D or 3D representation of the person based on at least a second subset of images from the video sequence, wherein the second 2D or 3D representation of the person represents a second pose or a second body shape of the person in the medical environment, and wherein the second 2D or 3D representation of the person includes an adjustment to the first 2D or 3D representation of the person.

13. The method of claim 12, wherein the adjustment included in the second 2D or 3D representation of the person includes a depiction of a body part of the person that is missing from the first 2D or 3D representation of the person.

14. The method of claim 12, wherein the second 2D or 3D representation of the person is determined further based on the first 2D or 3D representation of the person.

15. The method of claim 14, wherein determining the second 2D or 3D representation of the person further based on the first 2D or 3D representation of the person comprises determining a body part of the person that is missing from the first 2D or 3D representation of the person, and reconstructing the body part in the second 2D or 3D representation of the person based on the second subset of images.

16. The method of claim 12, wherein at least one of the first 2D or 3D representation of the person or the second 2D or 3D representation of the person is determined based on a machine-learning (ML) model.

17. The method of claim 16, wherein the ML model implemented via a convolutional neural network or a recurrent neural network.

18. The method of claim 12, wherein at least one of the first 2D or 3D representation of the person or the second 2D or 3D representation of the person is determined based on multiple machine-learning (ML) models, a first one of the multiple ML models trained for predicting a representation of a first body part of the person based on the video sequence, a second one of the multiple ML models trained for predicting a representation of a second body part of the person based on the video sequence, and wherein the at least one of the first 2D or 3D representation of the person or the second 2D or 3D representation of the person is determined by combining at least the representation of the first body part of the person and the representation of the second body part of the person.

19. The method of claim 12, wherein at least one of the first 2D or 3D representation of the person or the second 2D or 3D representation of the person includes a 3D mesh model of the person that includes a first plurality of parameters associated with a pose of the person, the 3D mesh model further including a second plurality of parameters associated with a body shape of the person.

20. The method of claim 12, wherein the video sequence is captured by a single image capturing device that includes at least one of a red-green-blue (RGB) image sensor, a depth sensor, an infrared sensor, a radar sensor, or a pressure sensor.
